(19) United States
(12) Patent Application Publication
(10) Pub. No.: US 2007/0243237 A1
(43) Pub. Date: Oct. 18, 2007

(54) Antimicrobial thin film coating and method of forming the same

(76) Inventors: Mazen Khaled, Dhahran (SA); Basel Abu-Sharkh, Dhahran (SA)

Correspondence Address: LITMAN LAW OFFICES, LTD., P.O. BOX 15035, CRYSTAL CITY STATION, ARLINGTON, VA, 22215, US

(21) Appl. No.: 11/404,025

(22) Filed: Apr. 14, 2006

(51) Int. Cl. A61K 9/70; C09D 5/16

(52) U.S. Cl. 424/443; 523/122; 977/906

(57) ABSTRACT

The antimicrobial thin film coating is a polyelectrolyte complex film applied to a substrate, with the polyelectrolyte complex material having biocidal properties. The polyelectrolyte complex material is formed from a first polyelectrolyte material having a positive charge, which is applied to the substrate in the form of a solution, and a second polyelectrolyte material having a negative charge, which is applied to the substrate following application of the first material. The positively charged polyelectrolytes and the negatively charged polyelectrolytes arrange themselves into a polyelectrolyte complex, rather than an alternating multi-layer structure, due to the electrostatic attraction between particles, allowing for the formation of a thin film with optimal coverage of the substrate.

### BACKGROUND OF THE INVENTION

1. Field of the Invention

The present invention relates to an antimicrobial and biocidal thin film coating. The thin film coating is a polyelectrolyte complex film, which is applied to a substrate, with the polyelectrolyte complex material having biocidal properties. Particularly, the polyelectrolyte complex is formed from a first polyelectrolyte material having a positive electrical charge and a second polyelectrolyte material having a negative electrical charge, which are both applied to the substrate in solution.

2. Description of the Related Art

A wide variety of biocidal compounds have been deposited or grafted onto substrates, such as metal, wood, rubber, plastic and the like, utilizing a variety of chemical and physical methods. Since the discovery of bacteria and other harmful microbes, antimicrobial agents have been sought and utilized in order to disinfect surfaces. Phenol, halogen and aldehyde derivatives, antibiotics, cationic compounds, surfactants, guanide derivatives, and organic heavy metal compounds have all been commonly utilized as biocides.

Presently, synthetic polymer materials are of interest in the fields of manufacturing and industry. With the production of polymers, researching the antibiotic properties of these polymer materials is getting easier, more cost effective and more efficient. The introduction of biologically active groups to monomers, followed by their polymerization, is of particular interest in the field of biocides. Another biocidal method of interest is the immobilization of water-soluble, emulsible, or suspendible disinfectants onto macromolecular material surfaces.

Further, present research is directed towards the use of low molecular weight (MW) cationic biocides. In use, the target sites of the biocides are cytoplasmic membranes of bacterial cells. The biocides are adsorbed onto the bacterial cell surface; they diffuse through the cell wall; they bind to the cytoplasmic membrane; they disrupt the cytoplasmic membrane; K⁺ ions are released; and the cell dies.

Cationic polymers with quaternary ammonium or biguanide groups tend to exhibit higher antimicrobial activity than the corresponding low MW model compounds. The higher activities of polycationic compounds are generally interpreted as follows: The bacterial cell surface is negatively charged at a physiological pH, and cationic disinfectants are positively charged at this pH. The disinfectants are adsorbed onto the cell surfaces by electrostatic interaction. The adsorption of polycations onto the negatively charged cell surfaces is expected to take place to a greater extent than that of monomeric cations due to the larger charge density carried by the polycations. Binding to the cytoplasmic membrane is also expected to be facilitated by the polycations, compared with that by the monomeric cations, due to the presence of a large number of negatively charged species in the membrane. Thus, the disruption of the membrane and subsequent leakage of K⁺ ions and other cytoplasmic constituents would be enhanced by the polycations.

Further, it has been found that the antibacterial activity of cationic disinfectants may be ascribed essentially to molecular organizations of cations within aggregates; i.e., the activity is determined by the size of aggregates and number of active molecules forming the aggregate. Thus, the morphological effect of disinfectants, which are low MW phosphonium salts with single and double long alkyl chains (C₁₄), in aqueous solution on the antibacterial activity may produce the lethal action of low MW cationic biocides. It has further been found that antibacterial activity is dependent on the size of the aggregates, and there further exists an optimal size range for the antibacterial activity of the cationic disinfectants. Similar properties have been found in cationic polymeric disinfectants.

The important factors involved with the use of cationic disinfectants include electrostatic interaction between the cells and the disinfectants and the hydrophobic moieties of the cationic disinfectants. After diffusion through the cell walls, the disinfectants need to have hydrophobic or lipophilic moieties in them, owing to their binding to the cytoplasmic membrane. Studies on antibacterial macromolecules have been made through use of syntheses or preparations of biologically active water soluble and insoluble macromolecules, immobilizations of biologically active groups onto macromolecular substrate surfaces, macromolecular films with antibacterial groups, as well as the antibacterial activities of the resulting macromolecules.

None of the above methods or systems, taken either singly or in combination, is seen to describe the instant invention as claimed. Thus, an antimicrobial thin film coating and method of forming the same solving the aforementioned problems is desired.

### SUMMARY OF THE INVENTION

The antimicrobial thin film coating is a polyelectrolyte complex film applied to a substrate, with the polyelectrolyte complex material having biocidal properties. The polyelectrolyte complex material is formed from a first polyelectrolyte material having a positive charge, which is applied to the substrate in the form of a solution, and a second polyelectrolyte material having a negative charge, which is applied to the substrate following application of the first material. Application may be through casting, dip coating, doctor blading, soaking, sedimenting, sprayings or combinations thereof, or the like.

The positively charged polyelectrolyte material may be a polyelectrolyte having a quaternary ammonium group, a polyelectrolyte having a pyridinium group, or a protonated polyamine. The negatively charged polyelectrolyte material may be a polyelectrolyte having a sulfonate group, a polycarboxylate or a polyphosphonic acid. Further, an additive may be added to the polyelectrolyte materials. The additive may be an organic material, an inorganic material, a metal, a nanoparticle material or a combination thereof.

The positively charged polyelectrolytes and the negatively charged polyelectrolytes arrange themselves into a polyelectrolyte complex, rather than an alternating multi-layer structure, due to the electrostatic attraction between particles, allowing for the formation of a thin film with optimal coverage of the substrate.

Alternatively, the substrate may be coated with a thin film coating formed from a first polyelectrolyte material having a first electric charge, and a second material having the opposite electric charge. Such a material could be, for example, a nanoparticle, such as a colloidal oxide.

Further, rather than coating the substrate with one polyelectrolyte material in solution, then the second polyelectrolyte material in solution, the two solutions could be mixed together, to form a polyelectrolyte complex precipitate. This precipitate is then removed and dissolved in a new solution, which is used to coat the substrate in order to grow the thin film.

These and other features of the present invention will become readily apparent upon further review of the following specification and drawings.

### BRIEF DESCRIPTION OF THE DRAWINGS

FIG. 1 is a polarogram showing adsorption characteristics of an antimicrobial thin film coating according to the present invention on stainless steel wire.

FIG. 2 is a side section view of the antimicrobial thin film coating according to the present invention applied to a substrate.

FIG. 3 is a UV-VIS absorption spectrum of a polystyrene sulfonate solution.

FIG. 4 is the UV-VIS absorption spectra of a multilayer polyelectrolyte system including polystyrene sulfonate and polydiallyldimethylammonium chloride deposited on glass, showing spectra after the application of each two layers.

FIG. 5 is the UV-VIS spectra of the polyelectrolyte system of FIG. 4, showing the strength of the spectra after a one-week interval in storage.

FIG. 6 is the UV-VIS spectrum of ciprofloxacin HCl.

FIG. 7 is a UV-VIS spectrum of a mixture of ciprofloxacin and polystyrene sulfonate.

FIG. 8 is the UV-VIS spectrum of a multilayer polyelectrolyte system including polystyrene sulfonate and polydiallyidimethylammonium chloride with ciprofloxacin admixed with the polystyrene sulfonate.

FIG. 9 is a polarogram showing adsorption characteristics of another antimicrobial thin film coating according to the present invention on stainless steel wire.

Similar reference characters denote corresponding features consistently throughout the attached drawings.

### DETAILED DESCRIPTION OF THE PREFERRED EMBODIMENTS

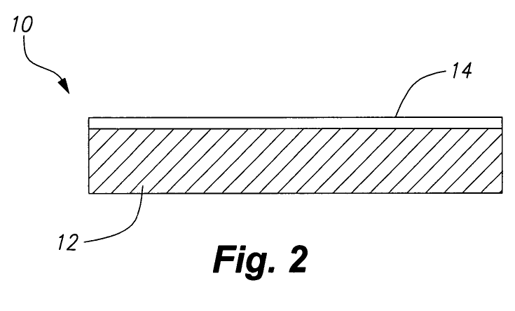

The present invention is directed to an antimicrobial thin film coating and a method of forming the coating. As shown in FIG. 2, a thin film layer 14 is formed on a substrate 12, with the coated substrate being designated generally as 10. The thin film layer 14 is an antimicrobial thin film coating formed of a polyelectrolyte complex having biocidal properties. Substrate 12 may be any substrate that the user wishes to coat with a biocidal thin film, such as, but not limited to: metals, including iron, steel, aluminum, magnesium, copper, titanium, beryllium, silicon, chromium, manganese, cobalt, nickel, palladium, lead, cerium, lithium, sodium, potassium, silver, cadmium, molybdenum, hafnium, antimony, tungsten, tantalum, vanadium, uranium, and mixtures and alloys thereof (e.g., stainless steel); polymers, including polyethylene, polypropylene, polyvinyl chloride, polystyrene, polybutadiene, neoprene, polyvinyl alcohol and mixtures thereof; textiles, such as cotton, wool, silk, polyester, polyacrylate, viscose, nylon and mixtures thereof; wood; ceramic materials; semiconducting materials; and any other materials forming a substrate that may be subject to attack by microorganisms, such as microbes, bacteria, viruses, fungi, parasites and the like.

The polyelectrolyte complex material is formed from a first polyelectrolyte material having a positive charge, which is applied to the substrate in the form of a solution, and a second polyelectrolyte material having a negative charge, which is applied to the substrate following application of the first material. Polyelectrolytes are charged polymers and may be soluble in water, soluble in an organic solvent, dispersed in water or dispersed in an organic solvent. The charged polymers comprise monomer units that are positively or negatively charged. The polyelectrolytes are macromolecules, each having a plurality of charged units arranged in a spatially regular or irregular manner.

The thin film layer in the present invention is formed from the alternating deposition of the oppositely charged polymers on the substrate. In the preferred embodiment, a buildup of multilayers is accomplished by alternate dipping; i.e., cycling a substrate between two reservoirs containing solutions (the solutions may be aqueous or non-aqueous) of polyelectrolytes of opposite charge, with a rinsing step in pure water following each immersion. Each cycle adds a layer of polymer via electrostatic forces to the oppositely charged surface and reverses the surface charge, thereby priming the film for the addition of the next layer.

Films prepared in this manner tend to be uniform, follow the contours and irregularities of the substrate, and have thicknesses of approximately 10 nm to 10,000 nm. The thickness of the films depends on many material-related factors, such as the preferred number of layers deposited, the ionic strength of the solutions, the types of polymers used, the deposition time, the deposition temperature and the solvents used.

Although studies have shown that the substantial interpenetration of the individual polymer layers results in little composition variation over the thickness of the film, these polymer thin films are, nonetheless, referred to as polyelectrolyte multilayers (PEMUs). Cationic polyelectrolytes are known to have antibacterial activity. PEMUs, though, have not been used as coatings for controlling microbial growth on surfaces. However, as will be described in greater detail below, the present inventors have discovered that PEMUs can be used to create ultrathin films or coatings that are effective in inhibiting microorganism growth. Further, the PEMUs are durable, have a prolonged effect, are relatively versatile and can be replenished using a relatively simple application process.

The polyelectrolytes may be synthetic, naturally occurring (such as proteins, enzymes, and polynucleic acids), or synthetically modified naturally occurring macromolecules, such as modified celluloses and lignins. The polyelectrolytes utilized in the formation of coated substrate 10 may be copolymers having a combination of charged and/or neutral monomers (i.e., positive and neutral; negative and neutral; positive and negative; or positive, negative, and neutral). Copolymers are macromolecules having a combination of two or more repeat units. Regardless of the exact combination of charged and neutral monomers, a polyelectrolyte used in the formation of the thin film coating of the present invention is predominantly positively charged or predominantly negatively charged.

The molecular weight of a synthetic polyelectrolyte molecule is typically on the order of between 100 and 10,000,000 grams/mole and, in the preferred embodiment, is approximately between 10,000 and 1,000,000 grams/mole. The molecular weight of naturally occurring polyelectrolyte molecules, such as biomolecules, can reach as high as approximately 10,000,000 grams/mole. The polyelectrolyte typically forms approximately between 0.01% and 40% by weight of a polyelectrolyte solution, and preferably is between 0.1% and 10% by weight.

The charged polyelectrolytes may be linear polyelectrolytes, branched polyelectrolytes, dendritic polyelectrolytes, graft polyelectrolytes, or the copolymers and block copolymers thereof.

Examples of negatively charged polyelectrolytes include polyelectrolytes having a sulfonate group (SO₃), such as poly(styrenesulfonic acid) (PSS), poly(2-acrylamido-2-methyl-1-propane sulfonic acid) (PAMPS), sulfonated poly(ether ether ketone) (SPEEK), sulfonated lignin, poly(ethylenesulfonic acid), poly(methacryloxyethylsulfonic acid), their salts and copolymers thereof; polycarboxylates, such as poly(acrylic acid) (PAA) and poly(methacrylic acid) and polyphosphonic acids.

Examples of positively charged polyelectrolytes include polyelectrolytes having a quaternary ammonium group, such as poly(diallyidimethylammonium chloride) (PDAD), poly(vinylbenzyltrimethyl-ammonium) (PVBTA), ionenes, poly(acryloxyethyltrimethyl ammonium chloride), poly(methacryloxy(2-hydroxy) propyltri methyl ammoniu m chloride, and copolymers thereof; polyelectrolytes including a pyridinium group, such as poly(N-methylvinylpyridine) (PMVP), other poly(N-alkylvinylpyridines), and copolymers thereof; and protonated polyamines, such as poly(allylaminehydrochloride) (PAH) and polyethyleneimmine (PEI).

The polyelectrolyte solution includes a solvent in which the selected polyelectrolyte is soluble. The solvent may be water or some other suitable organic or inorganic solvent. For example, poly(vinyl pyridine) alkylated with a methyl group (PNM4VP) is water soluble, whereas poly(vinyl pyridine) alkylated with an octyl group (PNO4VP) is organic solvent soluble.

Examples of polyelectrolytes used to form the antimicrobial thin film 14 that are soluble in water include poly(styrenesulfonic acid), poly(2-acrylamido-2-methyl-1-propane sulfonic acid), poly(acrylic acids), sulfonated lignin, poly(ethylenesulfonic acid), poly(methacryloxyethylsulfonic acid), poly(methacrylic acids), their salts and copolymers thereof; and poly(diallyldimethylammonium chloride), poly(vinylbenzyltrimethylammonium), ionenes, poly(acryloxyethyltrimethyl ammonium chloride), poly(methacryloxy(2-hydroxy)propyltrimethyl ammonium chloride), and copolymers thereof; and polyelectrolytes including a pyridinium group, such as poly(N-methylvinylpyridine), and protonated polyamines, such as PAH and PEI.

Examples of polyelectrolytes which are soluble in non-aqueous solvents, such as methanol, ethanol, dimethylformamide, acetonitrile, carbon tetrachloride, and methylene chloride include poly(N-alkylvinylpyridines), and copolymers thereof, where the alkyl group is longer than approximately four carbons. Other examples of polyelectrolytes soluble in organic solvents include poly(styrenesulfonic acid), SPEEK, sulfonated polyfluoroethylene, poly(2-acrylamido-2-methyl-1-propane sulfonic acid), poly(dial lyldimethylammoniu m chloride), poly(N-methylvinylpyridine) and poly(ethyleneimmine), where the small polymer counterion, for example, Na⁺, Cl⁻, or H⁺, has been replaced by a large hydrophobic counterion, such as tetrabutyl ammonium, tetrathethyl ammonium, iodine, hexafluorophosphate, tetrafluoroborate, or trifluoromethane sulfonate.

Additionally, the polyelectrolyte solutions may include one or more salts. A salt is a soluble, ionic, inorganic compound, which dissociates into stable ions in solution, such as sodium chloride. A salt included in the polyelectrolyte solutions may be used to control the thickness of the adsorbed layers. Specifically, the inclusion of a salt increases the thickness of the adsorbed polyelectrolyte layer. Preferably, the amount of the salt added to the polyelectrolyte solution is approximately 10% by weight or less.

The biocidal thin film coating is formed on the substrate through alternating application of the charged polyelectrolyte solutions. Rather than forming a first layer of, for example, positive polyelectrolytes, then a second layer of negative polyelectrolytes, then a third positive layer, and so on, the charged polymeric components interdiffuse and mix on the molecular level upon incorporation into the thin film. Due to the electrostatic attraction between the positive polyelectrolytes and the negative polyelectrolytes, the polyelectrolytes arrange themselves into a relatively unitary thin film layer, rather than forming layers of alternating charge. The polymeric components form a polyelectrolyte complex, which is a true molecular blend of the individual polymeric components.

The polyelectrolyte complex is formed and held together by the strong electrostatic complexation between the positive and negative polymer segments. The complexed polyelectrolyte within the thin film layer has the same amorphous morphology as a polyelectrolyte complex formed by mixing aqueous solutions of positive and negative polyelectrolyte. The arrangement of positive and negative polyelectrolyte molecules, which arrange themselves to achieve the lowest possible potential energy, provides for optimal coating of the thin film on the substrate.

Alternatively, the microbial coating may be applied to the substrate as a pre-formed polyelectrolyte complex. The pre-formed complex is formed from the mixing of oppositely charged polyelectrolytes in solution to form a polyelectrolyte complex precipitate, which is then dissolved or resuspended in a suitable solvent or liquid medium to form a polyelectrolyte complex solution or dispersion. The polyelectrolyte complex solution or dispersion is then applied to the substrate surface and the solvent or liquid is evaporated, leaving behind a thin film formed of the polyelectrolyte complex.

The polyelectrolyte solutions or the polyelectrolyte complex solution, or the related dispersions, are preferably deposited on the substrate through spraying or dip coating. However, application may further be through casting, doctor blading, soaking, sedimenting or combinations thereof, or through any other suitable method of application or deposition. Spraying is particularly preferred when applying the thin film coating via alternating exposure of oppositely charged polyelectrolyte solutions, or a single coat composed of one polyelectrolyte or a mixture of the polyelectrolytes.

The duration in which a polyelectrolyte solution is typically in contact with the substrate surface (i.e., the contact time) can vary from approximately a few seconds to several minutes in order to achieve optimal thickness. The time varies dependent upon how thick the user requires the thin film to be, and which materials are used in forming the thin film layer. Generally, a contact time of approximately 10 seconds provides a suitable thickness, particularly when sufficient agitation is provided.

The oppositely charged polyelectrolyte solutions may be applied to the substrate immediately after one another or, alternatively, after intermediate rinsing with a suitable solvent. Further, an additional drying step may be added between depositions.

Alternatively, a variety of additives may be incorporated into the thin film layer as it is formed. Such additives include inorganic materials, such as metallic oxide particles (e.g., silicon dioxide, aluminum oxide, titanium dioxide, iron oxide, zirconium oxide and vanadium oxide); metals; or organic and/or inorganic biocides. For example, nanoparticles of zinc, zinc oxide or zirconium oxide may be added to a polyelectrolyte solution or polyelectrolyte complex solution in order to improve the abrasion resistance of the deposited film.

Alternatively, the substrate may be coated with a thin film coating formed from a first polyelectrolyte material having a first electric charge, and a second material having the opposite electric charge formed as a surface charge. Such a material could be, for example, a nanoparticle, such as a colloidal oxide. Typically, the surface charge is negative and the particle, therefore, substitutes for a negative polyelectrolyte. These particles typically have a diameter of between approximately 1 nm and 1000 nm. In the preferred embodiment, the particles have diameters of approximately between 5 nm and 100 nm.

As an example of the formation method, stainless steel wires (type 316) having diameters of 1.35 mm were abraded with emery polishing paper, and then washed with deionized water. Some of the abraded wires were tested uncoated and anti-bacterial coatings were deposited on other abraded wires through spin coating with alternating oppositely charged polyelectrolyte solutions. One particular coating was formed from 0.1% polyacrylic acid having a molecular weight of 100,000 and antibiotic Erythromycin Ethylsuccinate, in 0.7M NaCl.

The uncoated and coated wires were placed in an electrochemical cell to test the electrochemical properties of the polyelectrolyte films in order to ensure that the film adhered to the substrate and affected the surface properties of the type 316 stainless steel. The electrochemical cell was run at room temperature. The electrolyte was 0.7M NaCI and the surface area of the wire dipped into the electrolyte was approximately 0.6 cm². The anodic polarization curves were recorded using an EG & G® Princeton Applied Research 273 potentiostat. The reference electrode was an Ag/AgCl electrode, against which all potentials were based.

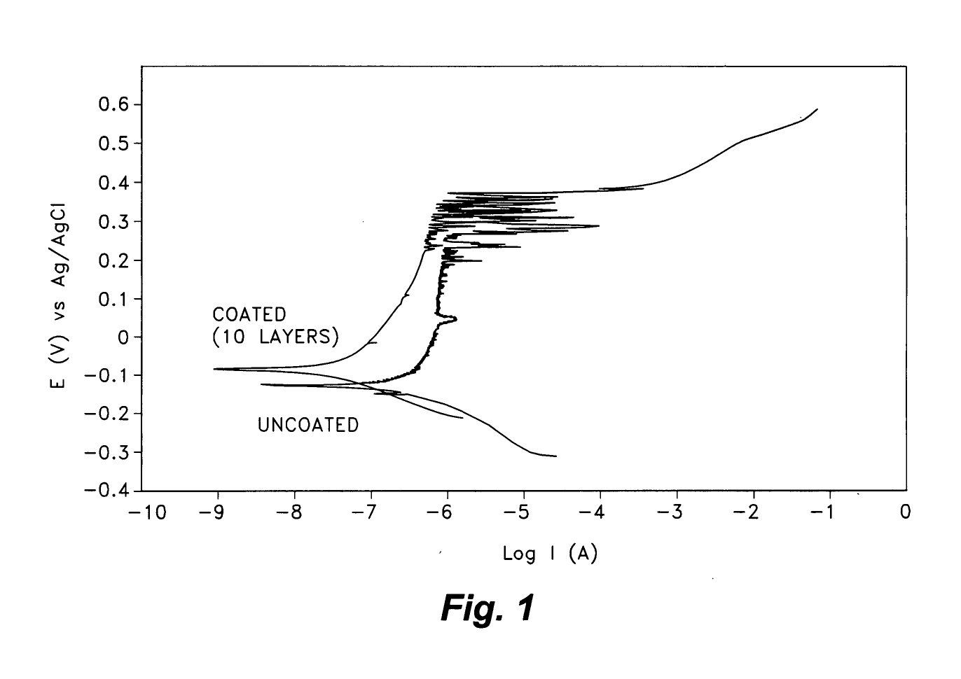

As illustrated in FIG. 1, the stainless steel wires were scanned from −150 mV vs. open circuit potential to 0.4 V. The plot of the uncoated stainless steel contains far more random current spikes than that of the coated material, indicating metastable pitting. The open circuit potential of the stainless steel coated with the antibiotic coating shifted to a more noble potential and the stainless steel exhibited fewer passive current densities, indicating the polyelectrolyte multilayer adheres to, and modifies, the surface properties of the stainless steel.

Further, the multilayer containing the antibiotic was placed in a solution containing staphylococcus aureus bacteria. Subsequent investigation of the solution indicated a decrease in growth of the bacteria population compared to a solution containing an uncoated surface.

The biocide functional groups bonded chemically or physically in the polymer structure, as an auxiliary biocide to the thin film, may include sulfa drugs, penicillin, cephalosporins, tetracycline, fluoroquinolones, nucleoside analogs, reverse transcriptase, protease inhibitors, halogens, phenolics, chlorhexidine, alcohols, hydrogen peroxide, heavy metals, aldehydes, quaternary ammonia compounds, quaternary imidazolium and pyrrolidinonium compounds, b-lacatamse, b-lactomines, cephaloporines, aminoglycosides, macrolides, fusidic acid, lincocemides, rifamycin, rifampicin tetracycline, chloremphenicicole, metronidazole, vancomycin, trimethoprime, sulfamethoxazole, novobiocine, fosfomycine, polymyxines, oxytetracycline, carbenicilin, ticarcillin, methiciillin, ampicillin, penicillin, aminobenzoic acids, sulfanilamides, cloxacillin, oxytetracycline, acryloxy and acryl groups, acrylates, sulfonamides, ascaphins, diterpenes and biguanide in their monomer or polymeric form, or may be sandwiched in the multilayer system. These biocidal functional groups may be formed in the polymer physically, chemically, through deposition, coating, precipitation, encapsulation, grafting, copolymerization or the like. The above listing of biocide functional groups is a representative listing only, and other biocide functional groups may be incorporated into the thin film of the present invention.

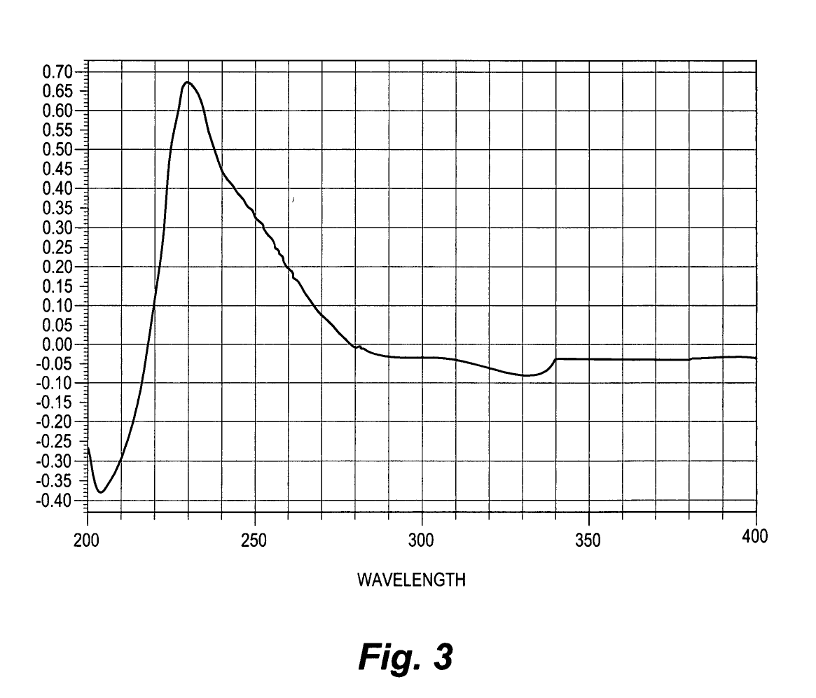

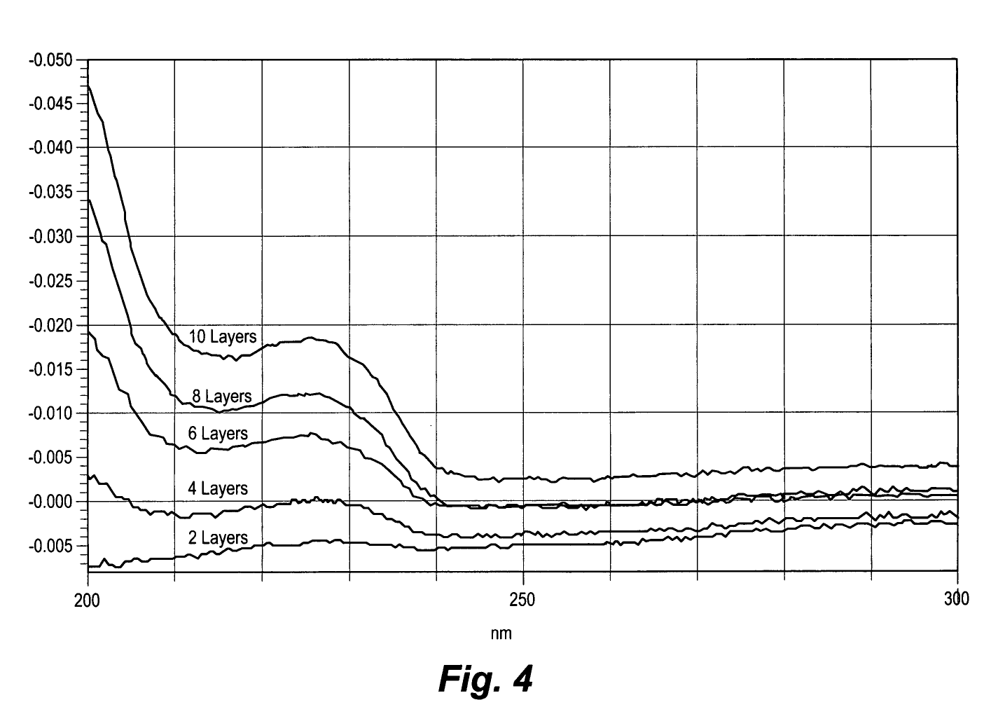

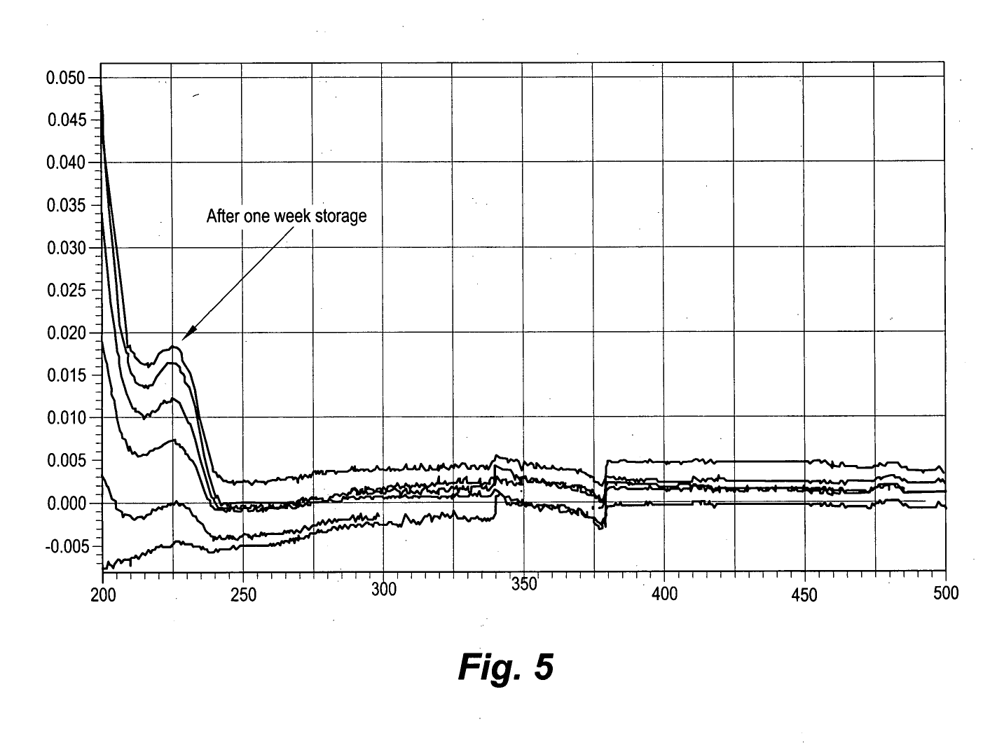

With regard to FIGS. 3 and 4, FIG. 3 illustrates the spectrum of 0.01% polystyrene sulfonate (sulfonated polystyrene) solution. The spectrum is produced by ultraviolet-visible light (UV-VIS) spectrometry. It should be noted that a maximum occurs at a wavelength of approximately 225 nm. FIG. 4 illustrates the spectra of a multilayer system deposited on glass following the alternating deposition of two layers each of 0.01% polystyrene sulfonate and polydimethyldiallylammonium chloride (PDADMAC). PDADMAC is a common antibacterial agent. The peak in the region of 225 nm increases as the number of multilayers increases. The peak at 225 nm corresponds to the peak at 225 nm shown in FIG. 3, the increase in absorbance showing that the concentration of polystyrene sulfonate increases with the increasing number of layers. FIG. 5 illustrates this same spectra measured after the substance and substrate were stored for one week. The peak still persists at 225 nm in FIG. 5, illustrating the stability of the antimicrobial film.

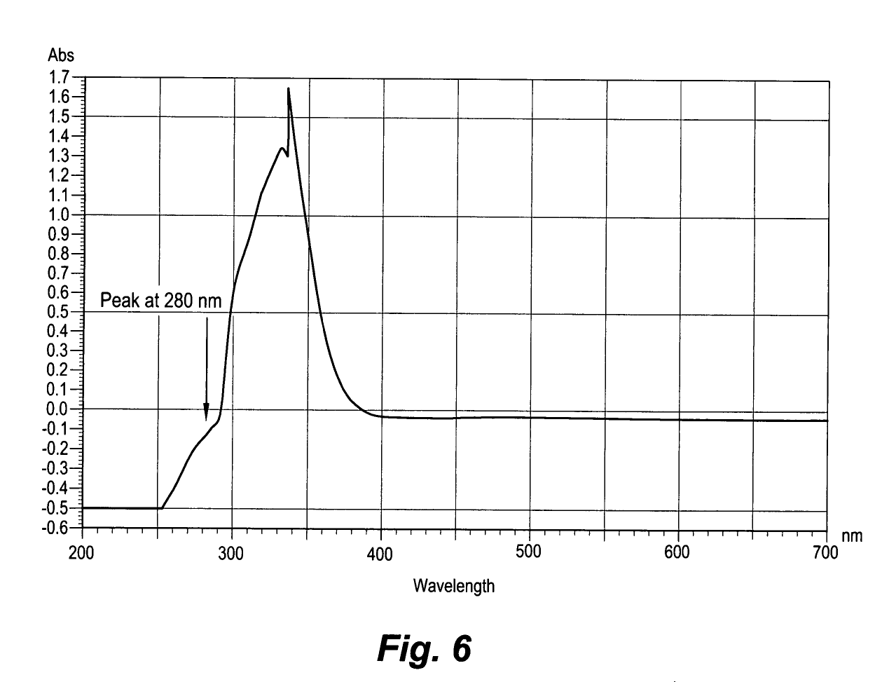

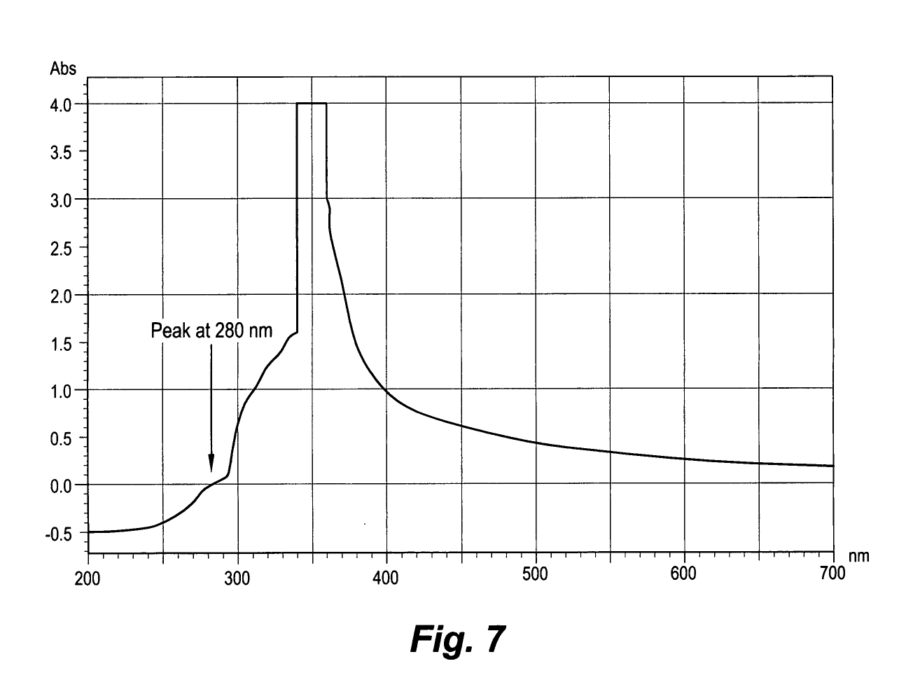

FIG. 6 illustrates the spectrum of a 0.01% solution of ciprofloxacin HCl, which is a common antibiotic having a molecular structure of C₁₇H₁₈FN₃O₃ HCl H₂O. FIG. 7 illustrates the spectrum of a solution mixture of 0.05% polystyrene sulfonate and 0.025% ciprofloxacin in solution. The spectrum includes a measured peak at 280 nm.

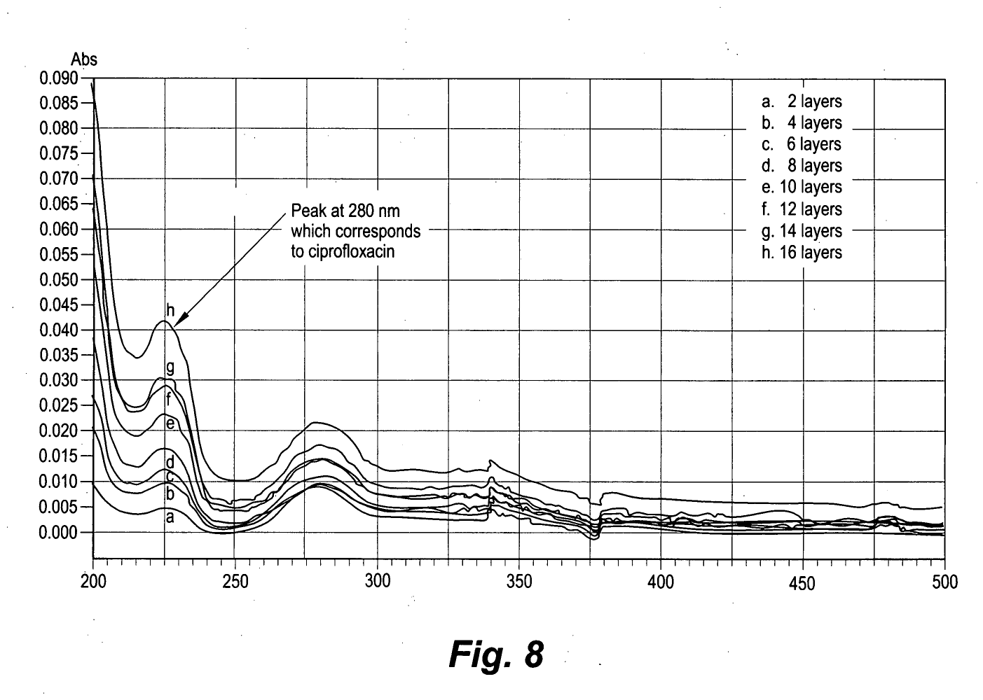

FIG. 8 illustrates the spectrum of a multilayer system formed by the deposition of a solution containing 0.01% PDAMAC and a solution containing a mixture of 0.0025% polystyrene sulfonated and 0.05% ciprofloxacin. The spectra shows that ciprofloxacin is encapsulated in the polyelectrolyte system, and the peak at 280 nm increases with increasing multilayers, showing increasing concentration of the biocide with the deposition of increasing layers of polyelectrolytes. Thus, antibiotics can be sandwiched in the polyelectrolyte layers. Such a sandwiched structure may be applied to a wide variety of antibiotics and mixtures of antibiotics, or to mixtures of antibacterial polyelectrolyte materials.

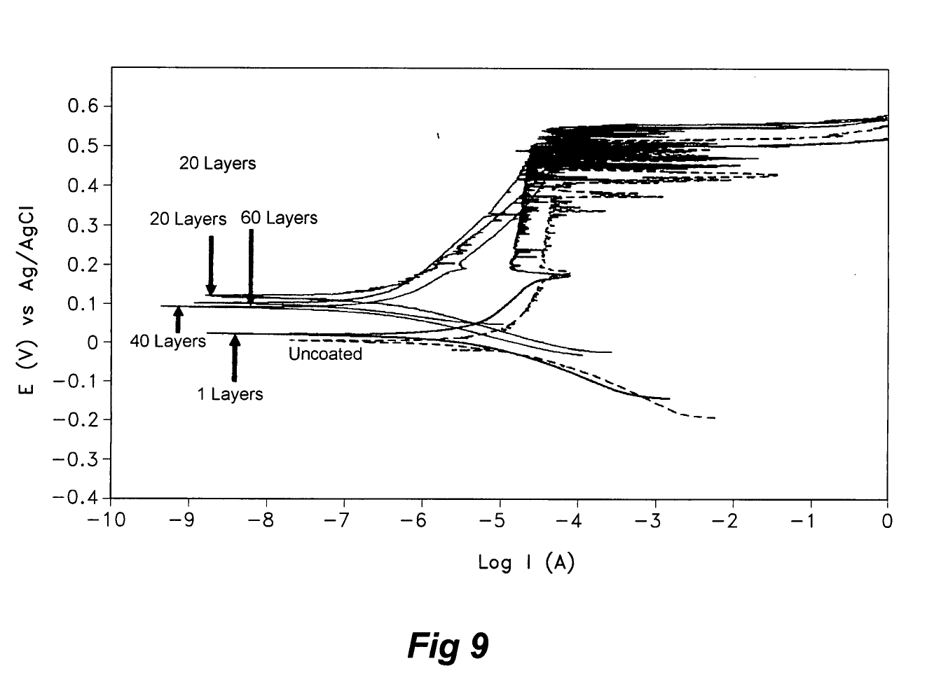

FIG. 9 illustrates an electrochemical test of the multilayer system deposited on 316 stainless steel and tested in a similar manner to that shown in FIG. 1, using polyacrylic acid and polyacrylamide. FIG. 9 illustrates an increase in the corrosion potential and a suppression of current. However, no significant change is shown after deposition of a 20 layers of polyelectrolytes.

It is to be understood that the present invention is not limited to the embodiments described above, but encompasses any and all embodiments within the scope of the following claims.

We claim:

1. An antimicrobial thin film coating, comprising:
a positively charged polyelectrolyte; and
a negatively charged polyelectrolyte, at least one of said polyelectrolytes having antimicrobial properties, the polyelectrolytes being formulated into a polyelectrolyte complex thin film adapted for coating a substrate.

2. The antimicrobial thin film coating as recited in claim 1, wherein said positively charged polyelectrolyte is a polyelectrolyte selected from the group consisting of polyelectrolytes having a quaternary ammonium group, polyelectrolytes having a pyridinium group, and protonated polyamines.

3. The antimicrobial thin film coating as recited in claim 2, wherein said negatively charged polyelectrolyte is a polyelectrolyte selected from the group consisting of sulfonated polyelectrolytes, polycarboxylates, and polyphosphonic acids.

4. The antimicrobial thin film coating as recited in claim 1, further comprising nanoparticles of an abrasion resistant material, the abrasion resistant material being selected from the group consisting of zinc, zinc oxide, and zirconium oxide.

5. The antimicrobial thin film according to claim 1, wherein said thin film has a thickness of between about 10 nm to 10,000 nm.

6. The antimicrobial thin-film according to claim 1, further comprising an effective amount of a salt for controlling the thickness of the film.

7. An antimicrobial thin film coating, comprising:
a polyelectrolyte having a first electrical charge associated therewith; and
an electrolyte bearing a surface electrical charge opposite in polarity to the first electrical charge, the polyelectrolyte and the electrolyte being formulated into a thin film adapted for coating on a substrate, the thin film having antimicrobial properties.

8. The antimicrobial thin film coating as recited in claim 7, wherein said electrolyte comprises nanoparticles.

9. The antimicrobial thin film coating as recited in claim 8, wherein said nanoparticles comprise nanoparticles of a colloidal oxide.

10. An antimicrobial thin film coating, comprising:
a positively charged polyelectrolyte;
a negatively charged polyelectrolyte, the polyelectrolytes being formulated into a polyelectrolyte complex thin film adapted for coating a substrate; and
a biocidal agent having antimicrobial properties, the biocidal agent being carried in said thin film.

11. The antimicrobial thin film coating according to claim 10, wherein said biocidal agent comprises at least one biocide selected from the group consisting of sulfa drugs, penicillin, cephalosporins, tetracycline, fluoroquinolones, nucleoside analogs, reverse transcriptase, protease inhibitors, halogens, phenolics, chlorhexidine, alcohols, hydrogen peroxide, heavy metals, aldehydes, quaternary ammonia compounds, quaternary imidazolium and pyrrolidinonium compounds, b-lacatamse, b-lactomines, cephaloporines, aminoglycosides, macrolides, fusidic acid, lincocemides, rifamycin, rifampicin tetracycline, chloremphenicicole, metronidazole, vancomycin, trimethoprime, sulfamethoxazole, novobiocine, fosfomycine, polymyxines, oxytetracycline, carbenicilin, ticarcillin, methiciillin, ampicillin, penicillin, aminobenzoic acids, sulfanilamides, cloxacillin, oxytetracycline, acryloxy and acryl groups, acrylates, sulfonamides, ascaphins, diterpenes and biguanide.

12. A method of forming an antimicrobial thin film coating, comprising the steps of:
(a) selecting a positively charged polyelectrolyte solution and a negatively charged polyelectrolyte solution adapted to form a polyelectrolyte complex upon mixing, at least one of the polyelectrolytes having antimicrobial properties;
(b) applying the positively charged polyelectrolyte solution to a substrate; and
(c) applying the negatively charged polyelectrolyte solution to the substrate to form a polyelectrolyte complex thin film.

13. The method of forming an antimicrobial thin film coating as recited in claim 12, wherein said applying steps comprises at least one applying step selected from the group consisting of casting, dip coating, doctor blading, soaking, sedimenting, and spraying.

14. The method of forming an antimicrobial thin film coating as recited in claim 12, further comprising repeating steps (b) and (c) to obtain a film thickness of between about 10 nm and 10,000 nm.

15. The method of forming an antimicrobial thin film coating as recited in claim 12, further comprising the step of rinsing said substrate with a solvent following said step of applying said positively charged polyelectrolyte solution and prior to said step of applying said negatively charged polyelectrolyte solution.

16. The method of forming an antimicrobial thin film coating as recited in claim 12, further comprising the steps of:
drying said polyelectrolyte complex thin film; and
reapplying said positively and negatively charged polyelectrolyte solutions in order to obtain a desired coating thickness.

17. A method of forming an antimicrobial thin film coating, comprising the steps of:
selecting a positively charged polyelectrolyte and a negatively charged polyelectrolyte solution adapted to form a polyelectrolyte complex upon mixing, at least one of the polyelectrolytes having antimicrobial properties
mixing said positively and negatively charged polyelectrolyte solutions to form a mixed solution and a polyelectrolyte complex precipitate;
removing said polyelectrolyte complex precipitate from said mixed solution;
providing a solvent;
dissolving said polyelectrolyte complex precipitate in said solvent to form a polyelectrolyte complex solution;
applying said polyelectrolyte complex solution to a substrate to form a polyelectrolyte complex thin film; and
reapplying said polyelectrolyte complex solution to said substrate to obtain a film thickness between about 10 nm and 10,000 nm.

18. The method of forming an antimicrobial thin film coating as recited in claim 17, wherein said applying step comprises at least one applying step selected from the group consisting of casting, dip coating, doctor blading, soaking, sedimenting, and spraying.

19. The method of forming an antimicrobial thin film coating as recited in claim 17, further comprising the step of rinsing said substrate with a second solvent following said step of applying said polyelectrolyte complex solution.

20. The method of forming an antimicrobial thin film coating as recited in claim 17, further comprising the step of drying said polyelectrolyte complex thin film and said substrate prior to said step of reapplying said polyelectrolyte complex solution.
